(19) United States
(12) Patent Application Publication
(10) Pub. No.: US 2025/0095147 A1
(43) Pub. Date: Mar. 20, 2025

(54) METHOD AND APPARATUS FOR DETERMINING ORIENTATION OF SUBJECT DURING MEDICAL IMAGING AND MEDICAL IMAGING SYSTEM

(71) Applicant: GE Precision Healthcare LLC, Waukesha, WI (US)

(72) Inventors: Ting Ye, Beijing (CN); Xiaolan Liu, Beijing (CN); Qingyu Dai, Beijing (CN); Hao Yang, Beijing (CN); Jian Cui, Beijing (CN); Yuyang Huang, Beijing (CN)

(21) Appl. No.: 18/889,344

(22) Filed: Sep. 18, 2024

(30) Foreign Application Priority Data
Sep. 20, 2023 (CN) 202311216207.8

(51) Int. Cl. G06T 7/00; A61B 90/00

(52) U.S. Cl. CPC G06T 7/0012; A61B 2090/364

(57) ABSTRACT

A method and apparatus for determining the orientation of a subject during medical imaging, and a medical imaging system is provided. The method includes: acquiring an image sequence of a subject captured by a camera, the image sequence comprising a plurality of images based on a time sequence; determining, based on the image sequence, whether the state of the subject is abnormal; and in response to a determination result that the state of the subject is abnormal, determining the orientation of the subject by using a first orientation or a second orientation, wherein the first orientation is the orientation of the subject determined based on an image before a period when the state of the subject is determined to be abnormal, and the second orientation is the orientation of the subject determined based on an image after the period when the state of the subject is determined to be abnormal.

### CROSS REFERENCE TO RELATED APPLICATIONS

The present application claims priority and benefit of Chinese Patent Application No. 202311216207.8 filed on Sep. 20, 2023, which is incorporated herein by reference in its entirety.

### TECHNICAL FIELD

Embodiments of the present application relate to the technical field of medical devices, and in particular to a method and apparatus for determining the orientation of a subject during medical imaging and a medical imaging system.

### BACKGROUND

In a scenario where a medical imaging system is used to scan and image a subject under detection, it is necessary to set the orientation of the subject under detection. When the actual orientation of the subject under detection is the same as the set orientation, the medical imaging system can perform correct scanning and imaging processing on the subject under detection.

The orientation of the subject under detection may include which part of the body enters the scanning space first. For example, a head-in type means that the head of the subject under detection enters the scanning space first, and a feet-in type means that the feet of the subject under detection enter the scanning space first. Detecting the orientation of the subject under detection may also include detecting which direction the face of the subject under detection faces towards, for example, detecting whether the subject under detection is prone or supine.

In a medical imaging system with a camera, an image of the subject under detection may be captured by the camera, and the orientation of the subject under detection may be determined based on the image.

It should be noted that the above introduction of the background is only for the convenience of clearly and completely describing the technical solutions of the present application, and for the convenience of understanding for those skilled in the art.

### SUMMARY

The inventors of the present application have found that when the orientation of a subject under detection is determined based on a current image of the subject under detection, if the state of the subject under detection is abnormal, the accuracy of a determination result of the orientation may be reduced. For example, when the subject under detection is blocked by foreign objects such as a blanket, a mat, or a coil, the camera cannot capture an image of a blocked part of the subject under detection, and it is difficult to extract key points accurately. Consequently, the orientation cannot be determined accurately. For another example, when the subject under detection is in a state of moving, the orientation cannot be determined accurately due to the possibility of changes in the orientation.

In order to solve the above technical problems or at least similar technical problems, provided in the embodiments of the present application are a method and apparatus for determining the orientation of a subject during medical imaging, and a medical imaging system. The method determines the orientation of the subject with reference to information of the time dimension. Therefore, the orientation of the subject can be determined more accurately.

According to an aspect of an embodiment of the present application, there is provided a method for determining the orientation of a subject during medical imaging. The method includes acquiring an image sequence of a subject captured by a camera, the image sequence comprising a plurality of images based on a time sequence; determining, based on the image sequence, whether the state of the subject is abnormal. The method further includes in response to a determination result that the state of the subject is abnormal, determining the orientation of the subject by using a first orientation or a second orientation. The first orientation is the orientation of the subject determined based on an image before a period when the state of the subject is determined to be abnormal, and the second orientation is the orientation of the subject determined based on an image after the period when the state of the subject is determined to be abnormal.

According to an aspect of an embodiment of the present application, there is provided an apparatus for determining the orientation of a subject during medical imaging. The apparatus includes an acquisition unit, which acquires an image sequence of a subject captured by a camera, the image sequence comprising a plurality of images based on a time sequence. The apparatus also includes a determination unit, which determines, based on the image sequence, whether the state of the subject is abnormal; and an orientation determination unit, which in response to a determination result that the state of the subject is abnormal, determines the orientation of the subject by using a first orientation or a second orientation. The first orientation is the orientation of the subject determined based on an image before a period when the state of the subject is determined to be abnormal, and the second orientation is the orientation of the subject determined based on an image after the period when the state of the subject is determined to be abnormal.

According to an aspect of an embodiment of the present application, there is provided a medical imaging system. The system includes: a controller, configured to perform the above-mentioned method for determining the orientation of the subject.

One of the beneficial effects of the examples of the present application is that: the orientation of the subject is determined according to the orientation before or after the period when the state of the subject is determined to be abnormal. Since the orientation of the subject is determined by using the information of the time dimension, the accuracy can be improved.

With reference to the following description and drawings, specific embodiments of the examples of the present application are disclosed in detail, and the means by which the principles of the examples of the present application can be employed are illustrated. It should be understood that the embodiments of the present application are therefore not limited in scope. Within the scope of the spirit and clauses of the appended claims, the embodiments of the present application include many changes, modifications, and equivalents.

### BRIEF DESCRIPTION OF THE DRAWINGS

The included drawings are used to provide further understanding of the examples of the present application, which constitute a part of the description and are used to illustrate the embodiments of the present application and explain the principles of the present application together with textual description. Evidently, the drawings in the following description are merely some examples of the present application, and a person of ordinary skill in the art may obtain other embodiments according to the drawings without involving inventive skill. In the drawings:

FIG. 1 is a schematic diagram of a magnetic resonance imaging system according to an embodiment of the present application;

FIG. 2 is a schematic diagram of a method for determining the orientation of a subject according to an embodiment of the present application;

FIG. 3 is a schematic diagram of a first orientation;

FIG. 4 is a schematic diagram of a second orientation;

FIG. 5 is a schematic diagram of operation 203;

FIG. 6 is a schematic flowchart of an example of determining the orientation of a subject according to an embodiment of the present application;

FIG. 7 is a schematic diagram of an apparatus for determining the orientation of a subject according to an embodiment of the present application; and

FIG. 8 is a schematic diagram of a medical imaging system.

### DETAILED DESCRIPTION

The foregoing and other features of the examples of the present application will become apparent from the following description and with reference to the drawings. In the description and drawings, specific embodiments of the present application are disclosed in detail, and part of the embodiments in which the principles of the examples of the present application may be employed are indicated. It should be understood that the present application is not limited to the described embodiments. On the contrary, the examples of the present application include all modifications, variations, and equivalents which fall within the scope of the appended claims.

In the embodiments of the present application, the terms “first” and “second” etc., are used to distinguish different elements, but do not represent a spatial arrangement or temporal order, etc., of these elements, and these elements should not be limited by these terms. The term “and/or” includes any and all combinations of one or more associated listed terms. The terms “comprise”, “include”, “have”, etc., refer to the presence of described features, elements, components, or assemblies, but do not exclude the presence or addition of one or more other features, elements, components, or assemblies.

In the embodiments of the present application, the singular forms “a” and “the”, etc., include plural forms, and should be broadly construed as “a type of” or “a class of” rather than being limited to the meaning of “one”. Furthermore, the term “the” should be construed as including both the singular and plural forms, unless otherwise specified in the context. In addition, the term “according to” should be construed as “at least in part according to . . . ” and the term “on the basis of” should be construed as “at least in part on the basis of . . . ”, unless otherwise specified in the context.

In the embodiments of the present application, the term “key point” may be equivalently replaced with “key coordinate point”, “landmark”, “landmark point”, or the like. The term “object” may be equivalently replaced with “object for detection”, “subject under detection”, “object being scanned”, “object to be scanned”, “patient”, “subject of study”, or the like, which may be a human being or an animal, or may be other objects.

In the embodiments of the present application, the term “include/comprise” when used herein refers to the presence of features, integrated components, steps, or assemblies, but does not preclude the presence or addition of one or more other features, integrated components, steps, or assemblies.

The features described and/or illustrated for one implementation may be used in one or more other implementations in the same or similar manner, be combined with features in other embodiments, or replace features in other implementations.

In the embodiments of the present application, a method for determining the orientation of a subject or an apparatus for determining the orientation of a subject may be applied to various medical imaging scenarios, including but not limited to magnetic resonance imaging (MRI), computed tomography (CT), ultrasound imaging, positron emission tomography (PET), single photon emission computed tomography (SPECT), PET/CT, PET/MR, or any other suitable medical imaging scenarios.

In the embodiments of the present application, the method, the apparatus and the system of the present application are exemplified by taking an MRI scenario as an example, and it can be understood that the contents of the embodiments of the present application also applies to other medical imaging scenarios.

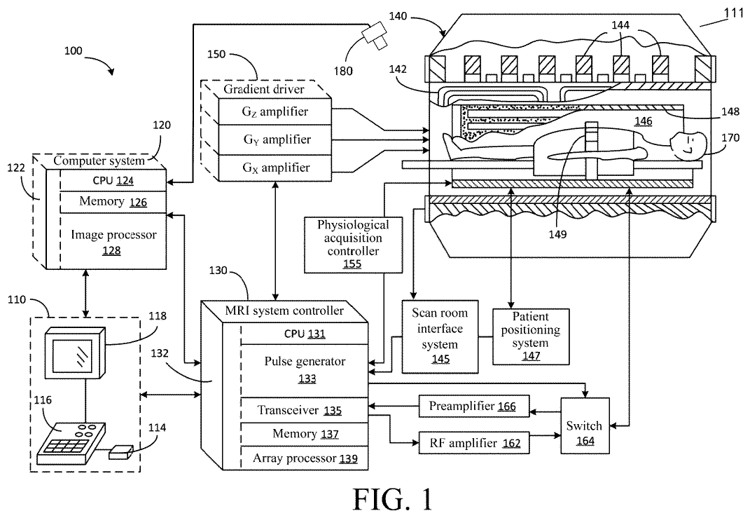

For ease of understanding, FIG. 1 is a schematic diagram of a magnetic resonance imaging (MRI) system 100 according to an embodiment of the present application.

The MRI system 100 includes a scanning unit 111. The scanning unit 111 is used to perform a magnetic resonance scan of a subject (e.g., a human body) 170 to generate image data of a region of interest of the subject 170, wherein the region of interest may be a pre-determined anatomical site or anatomical tissue.

The operation of the MRI system 100 is controlled by an operator workstation 110 that includes an input device 114, a control panel 116, and a display 118. The input device 114 may be a joystick, a keyboard, a mouse, a trackball, a touch-activated screen, voice control, or any similar or equivalent input apparatus. The control panel 116 may include a keyboard, a touch-activated screen, voice control, a button, a slider, or any similar or equivalent control device. The operator workstation 110 is coupled to and in communication with a computer system 120 that enables an operator to control the generation and display of images on the display 118. The computer system 120 includes various components that communicate with one another via an electrical and/or data connection module 122. The connection module 122 may employ a direct wired connection, a fiber optic connection, a wireless communication link, etc. The computer system 120 may include a central processing unit (CPU) 124, a memory 126, and an image processor 128. In some embodiments, the image processor 128 may be replaced by medical imaging functions implemented in the CPU 124. The computer system 120 may be connected to an archive media device, a persistent or backup memory, or a network. The computer system 120 may be coupled to and communicates with a separate MRI system controller 130.

The MRI system controller 130 includes a set of components that communicate with one another via an electrical and/or data connection module 132. The connection module 132 may employ a direct wired connection, a fiber optic connection, a wireless communication link, etc. The MRI system controller 130 may include a CPU 131, a sequence pulse generator (also known as a pulse generator) 133 in communication with the operator workstation 110, a transceiver (also known as an RF transceiver) 135, a memory 137, and an array processor 139.

In some embodiments, the sequence pulse generator 133 may be integrated into a resonance assembly 140 of the scanning unit 111 of the MRI system 100. The MRI system controller 130 may receive a command from the operator workstation 110, and is coupled to the scanning unit 111 to indicate an MRI scanning sequence to be performed during an MRI scan, so as to be used to control the scanning unit 111 to perform the flow of the aforementioned magnetic resonance scan. The MRI system controller 130 is further coupled to a gradient driver system (also known as gradient driver) 150 and is in communication therewith, and the gradient driver system is coupled to a gradient coil assembly 142 to generate a magnetic field gradient during an MRI scan.

The sequence pulse generator 133 may further receive data from a physiological acquisition controller 155 that receives signals from a plurality of different sensors (e.g., electrocardiogram (ECG) signals from electrodes attached to a patient, etc.), the sensors being connected to a subject or patient 170 undergoing an MRI scan. The sequence pulse generator 133 is coupled to and in communication with a scan room interface system 145 that receives signals from various sensors associated with the state of the resonance assembly 140. The scan room interface system 145 is further coupled to and in communication with a patient positioning system 147 that sends and receives signals to control movement of a patient table to a desired position to perform the MRI scan.

The MRI system controller 130 provides gradient waveforms to the gradient driver system 150, and the gradient driver system includes Gₓ (x direction), G_{y} (y direction), and G_{z} (z direction) amplifiers, etc. Each of the Gₓ, G_{y}, and G_{z} gradient amplifiers excites a corresponding gradient coil in the gradient coil assembly 142, so as to generate a magnetic field gradient used to spatially encode an MR signal during an MRI scan. The gradient coil assembly 142 is disposed within the resonance assembly 140, and the resonance assembly further includes a superconducting magnet having a superconducting coil 144 that, in operation, provides a static uniform longitudinal magnetic field B₀ throughout a cylindrical imaging volume 146. The resonance assembly 140 further includes an RF body coil 148, which, in operation, provides a transverse magnetic field B₁, and the transverse magnetic field B₁ is substantially perpendicular to B₀ throughout the entire cylindrical imaging volume 146. The resonance assembly 140 may further include an RF surface coil 149 for imaging different anatomical structures of the patient undergoing the MRI scan. The RF body coil 148 and the RF surface coil 149 may be configured to operate in a transmit and receive mode, a transmit mode, or a receive mode.

The x direction may also be referred to as a frequency encoding direction or a kₓ direction in the K-space, the y direction may be referred to as a phase encoding direction or a k_{y} direction in the K-space, and the Z direction can be referred to as a layer selection (layer selecting) direction. can be used for frequency encoding or signal readout, and is generally referred to as a frequency encoding gradient or a readout gradient. G_{y} G_{y} can be used for phase encoding, and is generally referred to as a phase encoding gradient. G_{z} can be used for slice (layer) position selection to acquire k-space data. It should be noted that a layer selection direction, a phase encoding direction, and a frequency encoding direction may be modified according to actual requirements.

The subject or patient 170 of the MRI scan may be positioned within the cylindrical imaging volume 146 of the resonance assembly 140. The transceiver 135 in the MRI system controller 130 generates RF excitation pulses amplified by an RF amplifier 162, and provides the same to the RF body coil 148 through a transmit/receive switch (also known as T/R switch or switch) 164.

As described above, the RF body coil 148 and the RF surface coil 149 may be used to transmit RF excitation pulses and/or receive resulting MR signals from the patient undergoing the MRI scan. The MR signals emitted by excited nuclei in the patient of the MRI scan may be sensed and received by the RF body coil 148 or the RF surface coil 149 and sent back to a preamplifier 166 through the T/R switch 164. The T/R switch 164 may be controlled by a signal from the sequence pulse generator 133 to electrically connect the RF amplifier 162 to the RF body coil 148 in the transmit mode and to connect the preamplifier 166 to the RF body coil 148 in the receive mode. The T/R switch 164 may further enable the RF surface coil 149 to be used in the transmit mode or the receive mode.

In some embodiments, the MR signals sensed and received by the RF body coil 148 or the RF surface coil 149 and amplified by the preamplifier 166 are stored in the memory 137 for post-processing as a raw k-space data array. A reconstructed magnetic resonance image may be obtained by transforming/processing the stored raw k-space data.

In some embodiments, the MR signals sensed and received by the RF body coil 148 or the RF surface coil 149 and amplified by the preamplifier 166 are demodulated, filtered, and digitized in a receiving portion of the transceiver 135, and transmitted to the memory 137 in the MRI system controller 130. For each image that is to be reconstructed, the data is rearranged into separate k-space data arrays, each of the separate k-space data arrays is input into the array processor 139, and the array processor is operated to transform the data into an array of image data by means of a Fourier transform.

The array processor 139 uses transform methods, most commonly Fourier transform, to create images from the received MR signals. These images are transmitted to the computer system 120 and stored in the memory 126. In response to commands received from the operator workstation 110, the image data may be stored in a long-term memory, or may be further processed by the image processor 128 and transmitted to the operator workstation 110 for presentation on the display 118.

In various embodiments, components of the computer system 120 and the MRI system controller 130 may be implemented on the same computer system or on a plurality of computer systems. It should be understood that the MRI system 100 shown in FIG. 1 is intended for illustration. Suitable MRI systems may include more, fewer, and/or different components.

The MRI system controller 130 and the image processor 128 may separately or collectively include a computer processor and a storage medium. The storage medium records a predetermined data processing program to be executed by the computer processor. For example, the storage medium may store a program configured to implement scanning processing (such as a scan flow and an imaging sequence), image reconstruction, medical imaging, etc. For example, the storage medium may store a computer program configured to determine the orientation of a subject according to the embodiments of the present invention. The described storage medium may include, for example, a ROM, a floppy disk, a hard disk, an optical disk, a magneto-optical disk, a CD-ROM, or a non-volatile memory card.

The inventors have found that when the orientation of the subject is determined by using the images captured by the camera, in some medical scenarios, it is necessary to cover a surface of the subject with a blocking object (for example, a coil, a mat, or a blanket). For example, in the MRI scenario described above, it is necessary to cover the surface of the subject 170 with the RF surface coil 149 or the like. Since some regions of the subject are covered with the blocking object, the camera cannot acquire an image within the range of the covered regions, and it is difficult to extract accurate key points. Consequently, the orientation cannot be determined accurately. In addition, when the subject under detection is in a state of moving, the orientation is also unlikely to be determined accurately due to the possibility of changes in the orientation.

To solve at least one of the above problems, provided in the embodiments of the present application are a method and apparatus for determining the orientation of a subject during medical imaging, and a medical imaging system.

Description is made below in conjunction with the examples.

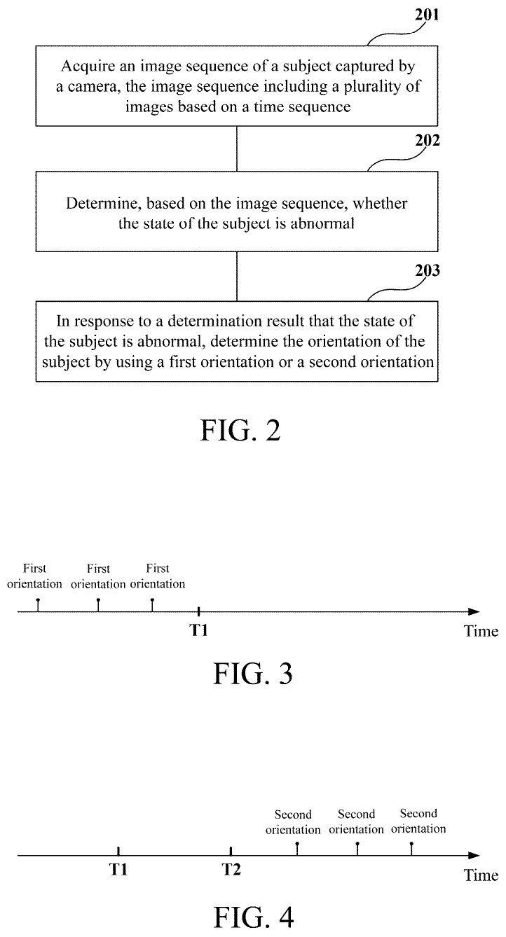

Provided in the embodiments of the present application is a method for determining the orientation of a subject during medical imaging. FIG. 2 is a schematic diagram of a method for determining the orientation of a subject during medical imaging according to an embodiment of the present application. As shown in FIG. 2, the method includes: at step 201: acquiring an image sequence of a subject captured by a camera, the image sequence including a plurality of images based on a time sequence. At step 202 it is determined whether the state of the subject is abnormal based on the image sequence. Finally at step 203: the orientation of the subject is determined by using a first orientation or a second orientation in response to a determination result that the state of the subject is abnormal.

In the present application, the first orientation is the orientation of the subject determined based on an image before a period when the state of the subject is determined to be abnormal, and the second orientation is the orientation of the subject determined based on an image after the period when the state of the subject is determined to be abnormal.

FIG. 3 is a schematic view of a first orientation, and FIG. 4 is a schematic view of a second orientation. As shown in FIG. 3, from time instance T1, it is determined that the state of the subject is abnormal, and the state may continue to be abnormal. Therefore, an image before a period when the state of the subject is determined to be abnormal may refer to an image before time instance T1. The subject orientation determined based on the image before time instance T1 is the first orientation. As shown in FIG. 4, from time instance T1, it is determined that the state of the subject is abnormal, and at time instance T2, it is determined that the state of the subject has returned from abnormal to normal. That is, the period when the state of the subject is determined to be abnormal refers to a period from time instance T1 to time instance T2. Therefore, an image after the period when the state of the subject is determined to be abnormal may refer to an image after time instance T2. The subject orientation determined based on an image after time instance T2 is the second orientation.

According to the above-mentioned embodiments, the orientation of the subject is determined according to the orientation before or after the period when the state of the subject is determined to be abnormal. Since the orientation of the subject is determined by using the information of the time dimension, the accuracy can be improved.

In some embodiments, the orientation of the subject under detection may include which part of the body enters the scanning space first. For example, a head-in type means that the head of the subject under detection enters the scanning space first, and a feet-in type means that the feet of the subject under detection enter the scanning space first. The orientation of the subject under detection may also include which direction the face of the subject under detection is towards, for example, whether the subject under detection is prone or supine.

In some embodiments, the image sequence may include a plurality of images, which are arranged into the image sequence in chronological order. Each image in the image sequence may include a color image and a depth image.

Each color image may include a plurality of pixel points, and a value of each pixel point may include a value of each color component. The color components may include a red (R) component, a green (G) component, a blue (B) component, and the like.

Each depth image may include a plurality of pixel points, and a value of each pixel point may include a depth value of the pixel. The depth value of the pixel may represent a distance from a point on the captured object that corresponds to the pixel to the camera.

In some embodiments, the camera may acquire a depth image sequence and a color image sequence during a period of time. The color image sequence may include a plurality of color images, and the depth image sequence may include a plurality of depth images. Each color image corresponds to a different time instance, and each depth image corresponds to a different time instance. The color images in the color image sequence may be in a one-to-one correspondence to the depth images in the depth image sequence. For example, the color images in the color image sequence may be aligned in time with the depth images in the depth image sequence. The present application is not limited thereto. The color images in the color image sequence may also be in other correspondence relationships with the depth images in the depth image sequence.

In some embodiments, the camera may be placed near (e.g., on an upper side of) the scanning unit 111 shown in FIG. 1, so as to capture the subject 170 from a top view and obtain an image. For example, it may be placed at the position of the camera 180 as shown in FIG. 1. In addition, the present application is not limited thereto, and the camera may also be disposed at other positions.

In the present application, the state of the subject being abnormal includes: the subject being blocked, and/or the subject moving. The subject being blocked refers to, for example, the subject being blocked by articles such as a coil or a blanket. The subject moving refers to at least a part of the body of the subject being in the state of moving, for example, the subject changing from a supine state to a prone state, or the subject getting up from a supine state to a sitting state. In addition, the state of the subject being abnormal may also refer to other situations, and the present application is not limited to the subject being blocked or the subject moving.

In operation 202, whether the state of the subject in the image is abnormal can be determined according to the depth image and/or the color image in the image sequence.

In some examples, whether the subject is blocked may be determined based on the depth image and the color image. For example, the following operations are adopted to determine whether the subject is blocked: a) operation 1: inputting a color image into a neural network, and outputting, by the neural network, a confidence level of each key point of the subject in the color image, where the confidence level may represent the possibility that the subject is blocked, for example, the lower the confidence level, the higher the possibility of being blocked; b) operation 2: using the depth image to obtain a depth value of each key point, where when the subject is blocked, a distance between a blocking object and the camera is short, so the depth value is small; and c) operation 3: when the confidence level of the key point is lower than a first threshold and the depth value of the key point is less than a second threshold, determining that the key point is blocked; and, when the number of key points determined to be blocked reaches a third threshold, determining that the subject is blocked.

In the present application, the key points may be, for example, feature points related to body parts that can be detected in an image. Position information of the key points may be represented by pixel positions in the image. The present application is not limited thereto. Position information of the key points may also be represented in other manners.

The types of the key points may include at least one of: head, breast, abdomen, neck, nose, left shoulder, right shoulder, left hip, right hip, left eye, right eye, left elbow, right elbow, left knee, right knee, left ear, right ear, left wrist, right wrist, left ankle, and right ankle. The present application is not limited thereto. The types of the key points may also include other contents.

The confidence level of the key point may be in the form of probability. For example, the confidence level of the key points refers to the probability that the actual position of the key point is located at the position obtained from detection. The present application is not limited thereto. The confidence level of the key point may also be in other forms or has other meanings.

In some other examples, whether the state of the subject is blocked may be determined based on the depth image. For example, the depth value of each key point is obtained by using the depth image. When the depth value of the key point is less than the second threshold, it is determined that the key point is blocked. When the number of the key points determined to be blocked reaches the third threshold, it is determined that the subject is blocked.

In still other examples, whether the state of the subject is blocked may be determined based on the color image. For example, the color image is input to the neural network, and the neural network outputs the confidence level of each key point of the subject in the color image. If the confidence level of the key point is lower than the first threshold, it is determined that the key point is blocked. In addition, when the number of the key points determined to be blocked reaches the third threshold, it is determined that the subject is blocked. The neural network may be trained, for example, in the following manners: an image set with known key points and key point confidence levels is taken as an input set, and the known key point confidence level is taken as an output set, to train the neural network.

In operation 202 of the present application, whether the subject is blocked may be determined based on the blocked state of the key points of the subject and time information of the blocked state. For example, Table 1 is an example of blocked state and time information of blocked state.

TABLE 1

| | Time | | Time | | Time |
|---|---|---|---|---|---|
| | instance 1 | . . . | instance p | . . . | instance M1 |
| Key point 1 | 1 | . . . | 0 | . . . | 1 |
| . . . | . . . | . . . | . . . | . . . | |
| Key point j | 1 | . . . | 1 | . . . | 0 |
| . . . | . . . | . . . | . . . | . . . | |
| Key point M2 | 1 | . . . | 0 | . . . | 0 |

As shown in Table 1, M1 is the number of color images or depth images, and the p-th image corresponds to time instance p in the time sequence; M2 is the total number of key points that can be detected by the neural network model, where 0 in the table indicates that the key point is blocked at a certain time instance p, and 1 in the table indicates that the key point is not blocked at the certain time instance p.

In the time sequence, if the first “0” appears at time instance T1, or the number of “0” appearing at the same time is greater than or equal to a first predetermined number, it is determined that the subject is blocked from time instance T1. After T1, if the last “0” appears at time instance T2, or the number of “0” appearing at the same time is less than or equal to a second predetermined number, it is determined that the state of the subject being blocked ends at time instance T2.

In some examples, the method for determining whether the subject is moving based on the depth image and/or the color image may be, for example: determining whether the depth value of the key point has changed based on the depth image, determining whether the position of the key point in the image has changed based on the color image, and determining that the subject is moving when at least one of the changes exceeds the corresponding threshold.

For example, based on the depth image, it is determined that changes in the depth values of the key points respectively corresponding to the left shoulder, the right shoulder, the left car, and the right car exceed the first threshold, and based on the color image, it is determined that changes in the positions of the key points corresponding to the left shoulder, the right shoulder, the left ear, and the right car in the image exceed the second threshold. Thus, the subject is determining to be moving.

The above examples serve merely for exemplifying, and the present application is not limited thereto. Other methods may also be adopted to determine whether or not a subject is abnormal.

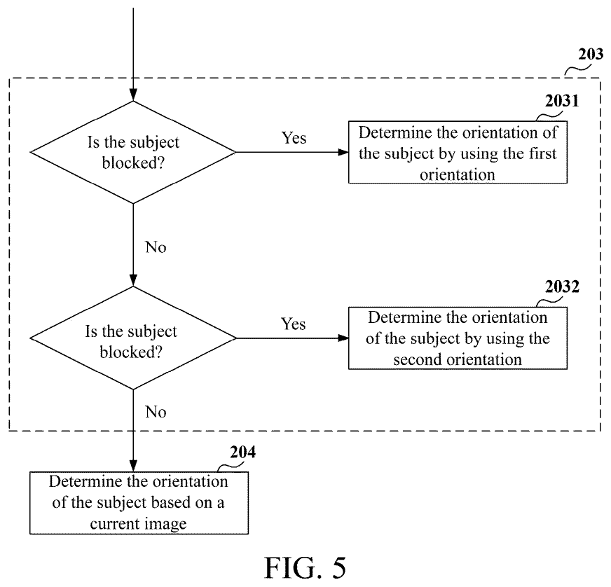

FIG. 5 is a schematic diagram of operation 203. As shown in FIG. 5, in operation 203, in response to a determination result that the state of the subject is abnormal (for example, the determination result obtained in operation 202), the orientation of the subject is determined by using the first orientation or the second orientation.

As shown in operation 2031 of FIG. 5, in at least some embodiments, in response to a determination result that the subject is blocked, the orientation of the subject is determined by using the first orientation. The determining the orientation of the subject by using the first orientation may include the following manners: a) manner 1: taking, as the orientation of the subject, the first orientation corresponding to a first predetermined image before the state of the subject is determined to be blocked; or b) manner 2: performing processing on the first orientations respectively corresponding to at least two images before the state of the subject is determined to be blocked to obtain the orientation of the subject.

With respect to manner 1, for example, it is determined that the subject is blocked from time instance T1, and therefore the first orientation corresponding to the first predetermined image before time instance T1 is taken as the orientation of the subject. The first predetermined image may be an image closest in time to time instance T1, or an image with a preset time interval from time instance T1.

With respect to manner 2, for example, it is determined that the subject is blocked from time instance T1, and therefore processing is performed on the first orientations respectively corresponding to at least two images before time instance T1 to obtain the orientation of the subject. The number of the at least two images may be a preset number, for example, N images before time instance T1. The processing includes at least one of the following processing.

Processing 1: performing processing of weighted summation on the first orientations respectively corresponding to the at least two images. For example, N images correspond to N first orientations, and different types of first orientations correspond to different values (for example, a value of the head-in supine type is 1, a value of the head-in prone type is 2, a value of the foot-in supine type is 3, . . . , and so on). In addition, the closer in time to time instance T1, the greater or less a weight assigned to the corresponding first orientation of the image is. Thus, the type of orientation that is closer to the value obtained by performing processing of weighted summation on the N first orientations is determined to be the orientation of the subject.

Processing 2: performing processing of averaging on the first orientations respectively corresponding to the at least two images. For example, different types of first orientations correspond to different values (for example, a value of the head-in supine type is 1, a value of the head-in prone type is 2, a value of the foot-in supine type is 3, . . . , and so on). Processing of averaging is performed on the N first orientations, and the type of orientation that is closer to the value obtained by the processing is determined to be the orientation of the subject.

Processing 3: processing of selecting the first orientation the number of occurrences of which is the greatest. For example, among N (e.g., N=5) first orientations, the head-in supine type appears 4 times. Then, the orientation of the head-in supine type is determined to be the orientation of the subject.

Processing 4: processing of selecting the first orientation the number of occurrences of which exceeds a threshold, where the threshold is 2, for example. Among N (for example, N=5) first orientations, the foot-in prone type appears 3 times (greater than the threshold), the head-in supine type appears once, and the head-in prone type appears once. Then, the orientation of the head-in supine type is determined to be the orientation of the subject. In addition, if the numbers of occurrences of more than two orientations exceed the threshold, at least one of processing 3, processing 2, or processing 1 may be further used to determine the orientation of the subject.

As shown in operation 2032 of FIG. 5, in at least some other embodiments, in response to a determination result that the subject is moving, the orientation of the subject is determined by using the second orientation. The determining the orientation of the subject by using the second orientation may include the following manners: c) manner 3: taking, as the orientation of the subject, the second orientation corresponding to a second predetermined image after the period when the state of the subject is determined to be moving; or, d) manner 4: performing processing on the second orientations respectively corresponding to at least two images after the period when the state of the subject is determined to be moving to obtain the orientation of the subject.

With respect to manner 3, for example, during the period from time instance T1 to time instance T2, it is determined that the subject is moving, and therefore the second orientation corresponding to the second predetermined image after time instance T2 is taken as the orientation of the subject. The second predetermined image may be an image closest in time to time instance T2, or an image with a preset time interval from time instance T2.

With respect to manner 4, for example, during the period from time instance T1 to time instance T2, it is determined that the subject is moving, and therefore processing is performed on the second orientations corresponding to at least two images after time instance T2 to obtain the orientation of the subject. The number of the at least two images may be a preset number, for example, M images after time instance T2. The processing includes at least one of the following processing c) Processing 5; f) Processing 6; g) Processing 7; h) Processing 8 as explained in detail below.

Processing 5: performing processing of weighted summation on the second orientations respectively corresponding to the at least two images. For example, M images correspond to M second orientations, and different types of second orientations correspond to different values (for example, a value of the head-in supine type is 1, a value of the head-in prone type is 2, a value of the foot-in supine type is 3, . . . , and so on). In addition, the closer in time to time instance T2, the greater or less a weight assigned to the corresponding second orientation of the image. Thus, the type of orientation that is closer to the value obtained by performing processing of weighted summation on the M second orientations is determined to be the orientation of the subject.

Processing 6: performing processing of averaging on the second orientations respectively corresponding to the at least two images. For example, different types of second orientations correspond to different values (for example, a value of the head-in supine type is 1, a value of the head-in prone type is 2, a value of the foot-in supine type is 3, . . . , and so on). Processing of averaging is performed on the M second orientations, and the type of orientation that is closer to the value obtained by the processing is determined to be the orientation of the subject.

Processing 7: processing of selecting the second orientation the number of occurrences of which is the greatest. For example, among M (e.g., M=5) second orientations, the head-in supine orientation appears 4 times. Then, the orientation of the head-in supine type is determined to be the orientation of the subject.

Processing 8: processing of selecting the second orientation the number of occurrences of which exceeds a threshold, where the threshold is 2, for example. Among M (for example, M=5) second orientations, the foot-in prone type appears 3 times (greater than the threshold), the head-in supine type appears once, and the head-in prone type appears once. Then, the orientation of the head-in supine type is determined to be the orientation of the subject. In addition, if the numbers of occurrences of more than two orientations exceed the threshold, at least one of processing 7, processing 6, or processing 5 may be further used to determine the orientation of the subject.

Furthermore, if the subject is detected to be moving during the period when the subject is blocked, the orientation of the subject may be determined based on manner 1 or manner 2. For example, if it is determined that the subject is blocked from time instance T1, and after time instance T1, the subject is detected to be moving during the period from time instance T11 to time instance T2, the first orientation corresponding to the first predetermined image before time instance T1 is taken as the orientation of the subject. Alternatively, processing is performed on the first orientations respectively corresponding to at least two images before time instance T1 to obtain the orientation of the subject.

In addition, as shown in FIG. 5, if the state of the subject is not abnormal, as shown in operation 204, the orientation of the subject is determined based on a current image.

In some embodiments of the present application, the first orientation or the second orientation may be obtained based on the depth image and/or the color image of the image.

For example, the color image may be input into an orientation determination model to obtain the orientation (such as the first orientation or the second orientation) corresponding to the color image. The orientation determination model may determine the orientation of the subject based on a classification network. Alternatively, the orientation determination model may detect the key points of the color image, and determine the orientation of the subject according to results of detecting the key points (for example, information such as the positions of the key points).

For another example, the depth information of the key points in the depth image may be compared with templates of depth information corresponding to different orientations, so that the orientation of the subject may be determined.

Reference may be made to the related art for the specific details of the method for determining the first orientation or the second orientation. In addition, the method for determining the first orientation or the second orientation may not be limited to the above description, and other methods may also be adopted.

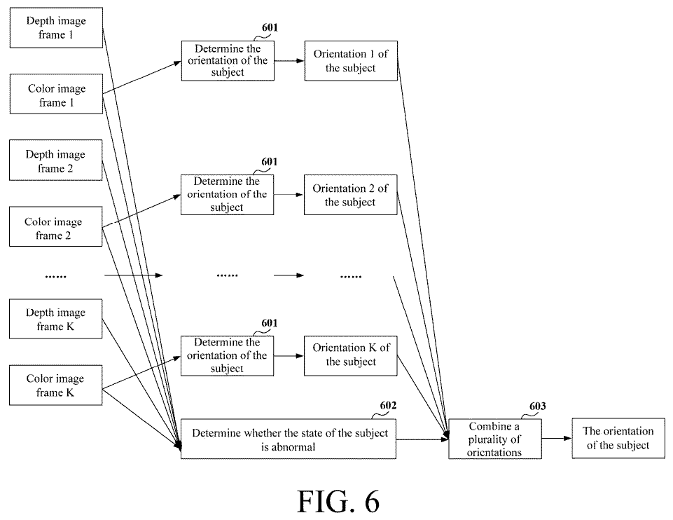

FIG. 6 is a schematic flowchart of an example of determining the orientation of a subject according to an embodiment of the present application.

As shown in FIG. 6, the image sequence includes depth image 1 to depth image K and color image 1 to color image K (where K is a natural number greater than or equal to 2). Depth image i and color image i correspond to the same time instance i (where i is a natural number greater than or equal to 1 and less than or equal to K).

In the example shown in FIG. 6, in operation 601, the orientation of the subject is determined according to each color image i, so that orientation i corresponding to each color image i, that is, orientation 1 to orientation K, is output.

As shown in FIG. 6, in operation 602, based on depth image i and color image i, whether the state of the subject is abnormal is determined at time instance i.

As shown in FIG. 6, in operation 603, a plurality of orientations i are combined, so that orientation information of the subject is output.

For example, when the state of the subject is not abnormal at time instance i, orientation i is output in operation 603. When the state of the subject is abnormal at time instance i, the orientation of the subject is determined by using the first orientation before time instance i in operation 603, or the orientation of the subject is determined by using the second orientation after the state of the subject returns to normal (that is, after the abnormality ends) in operation 603.

According to the above-mentioned embodiments, the orientation of the subject is determined according to the orientation before or after the period when the state of the subject is determined to be abnormal. Since the orientation of the subject is determined by using the information of the time dimension, the accuracy can be improved.

Further provided in the embodiments of the present application is an apparatus for determining the orientation of a subject, of which the same contents as those in the previous embodiments are not described again here.

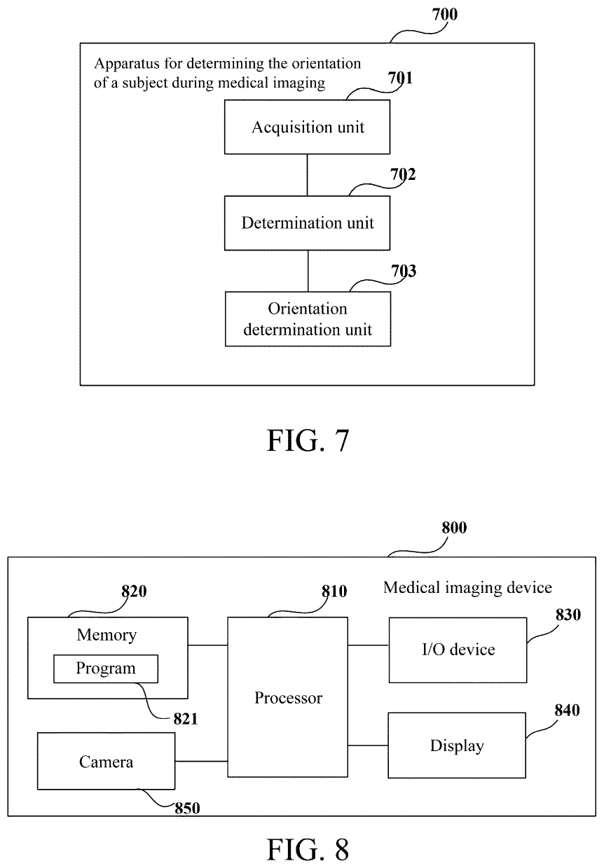

FIG. 7 is a schematic diagram of an apparatus for determining the orientation of a subject according to an embodiment of the present application. As shown in FIG. 7, the apparatus 700 for determining the orientation of the subject during medical imaging includes: an acquisition unit 701, which acquires an image sequence of a subject captured by a camera, the image sequence including a plurality of images based on a time sequence. The apparatus 700 further includes a determination unit 702, which determines, based on the image sequence, whether the state of the subject is abnormal; and an orientation determination unit 703, which in response to a determination result that the state of the subject is abnormal, determines the orientation of the subject by using a first orientation or a second orientation.

In some embodiments, the first orientation is the orientation of the subject determined based on an image before a period when the state of the subject is determined to be abnormal, and the second orientation is the orientation of the subject determined based on an image after the period when the state of the subject is determined to be abnormal.

In some embodiments, the abnormal state of the subject includes: the subject being blocked, and/or the subject moving.

In some embodiments, the orientation determination unit determines, in response to a determination result that the subject is blocked, the orientation of the subject by using the first orientation.

In some embodiments, the determining the orientation of the subject by using the first orientation includes: a) taking, as the orientation of the subject, the first orientation corresponding to a first predetermined image before the state of the subject is determined to be blocked; or b) performing processing on the first orientations respectively corresponding to at least two images before the state of the subject is determined to be blocked to obtain the orientation of the subject.

In some embodiments, the processing includes at least one of: performing, on the first orientations respectively corresponding to the at least two images, processing of weighted summation, processing of averaging, processing of selecting the first orientation the number of occurrences of which is the greatest, or processing of selecting the first orientation the number of occurrences of which exceeds a threshold.

In some embodiments, the orientation determination unit determines, in response to a determination result that the subject is moving, the orientation of the subject by using the second orientation.

In some embodiments, the determining the orientation of the subject by using the second orientation includes: a) taking, as the orientation of the subject, the second orientation corresponding to a second predetermined image after the period when the state of the subject is determined to be moving; or b) performing processing on the second orientations respectively corresponding to at least two images after the period when the state of the subject is determined to be moving to obtain the orientation of the subject.

In some embodiments, the processing includes at least one of: performing, on the second orientations respectively corresponding to the at least two images, processing of weighted summation, processing of averaging, processing of selecting the second orientation the number of occurrences of which is the greatest, or processing of selecting the second orientation the number of occurrences of which exceeds a threshold.

In some embodiments, each image in the image sequence includes a depth image and a color image.

The determination unit determines, based on the depth image and/or the color image, whether the state of the subject is abnormal.

In some embodiments, the first orientation or the second orientation is obtained based on the depth image and/or the color image of the image.

It is worth noting that only components or modules related to the present application have been described above, but the present application is not limited thereto. The apparatus 700 for determining the orientation of the subject may further include other components or modules. For the specifics of these components or modules, reference may be made to the related art.

For the sake of simplicity, FIG. 7 only exemplarily illustrates the connection relationship or signal direction between various components or modules, but it should be clear to those skilled in the art that various related technologies such as bus connection can be used. The various components or modules can be implemented by means of hardware such as a processor or a memory, etc. The embodiments of the present application are not limited thereto.

The above embodiments merely provide illustrative descriptions of the embodiments of the present application. However, the present application is not limited thereto, and appropriate variations may be made on the basis of the above embodiments. For example, each of the above embodiments may be used independently, or one or more among the above embodiments may be combined.

Further provided in the embodiments of the present application is a medical imaging system. The apparatus 700 for determining the orientation of a subject during medical imaging as described in the embodiment of the second aspect is included, the contents of which are incorporated here. The medical imaging device may, for example, have a computer, a server, a workstation, a laptop computer, a smart phone, or the like. However, the embodiments of the present application are not limited thereto.

FIG. 8 is a schematic diagram of a medical imaging device according to an embodiment of the present application. As shown in FIG. 8, a medical imaging device 800 may include: one or more processors (for example, central processing units (CPUs)) 810 and one or more memories 820. The memory 820 is coupled to the processor 810. The memory 820 may store various types of data. In addition, the memory further stores a program 821 for information processing, and executes the program 821 under the control of the processor 810.

In some embodiments, functions of the apparatus 700 for determining the orientation of a subject during medical imaging are implemented by integration into the processor 810. The processor 810 is configured to implement the method for determining the orientation of a subject during medical imaging described in the above embodiments of the present application.

In some embodiments, the apparatus 700 for determining the orientation of a subject during medical imaging is configured separately from the processor 810. For example, the apparatus 700 for determining the orientation of a subject during medical imaging may be configured as a chip connected to the processor 810, and the functions of the apparatus 700 for determining the orientation of a subject during medical imaging may be implemented by means of the control of the processor 810.

For example, the processor 810 is configured to perform the following control: acquiring an image sequence of a subject captured by a camera, the image sequence including a plurality of images based on a time sequence; determining, based on the image sequence, whether the state of the subject is abnormal; and in response to a determination result that the state of the subject is abnormal, determining the orientation of the subject by using a first orientation or a second orientation, wherein the first orientation is the orientation of the subject determined based on an image before a period when the state of the subject is determined to be abnormal, and the second orientation is the orientation of the subject determined based on an image after the period when the state of the subject is determined to be abnormal.

In a specific example, the medical imaging device 800 of FIG. 8 may be the magnetic resonance imaging (MRI) system 100 shown in FIG. 1. The memory 820 of FIG. 8 may correspond to at least one of the memory 137 and the memory 126 of FIG. 1. For example, the memory 820 may be independent of at least one of the memory 137 and the memory 126. Alternatively, the memory 820 may communicate with at least one of the memory 137 and the memory 126. Alternatively, the memory 820 may include at least one of the memory 137 and the memory 126. The processor 810 of FIG. 8 may correspond to at least one of the CPU 131, the CPU 124, and the image processor 128 of FIG. 1. For example, the processor 810 may be independent of at least one of the CPU 131, the CPU 124, and the image processor 128. Alternatively, the processor 810 may communicate with at least one of the CPU 131, the CPU 124, and the image processor 128. Alternatively, the processor 810 may include at least one of the CPU 131, the CPU 124, and the image processor 128.

In addition, as shown in FIG. 8, the medical imaging device 800 may further include: an input/output (I/O) device 830, a display 840, and the like. The functions of said components are similar to those in the prior art, and are not described again here.

In addition, as shown in FIG. 8, the medical imaging device 800 may further include a camera 850, which captures a subject and generates an image sequence. The image sequence may be transmitted to the processor 810, so that the processor 810 can implement the method for determining the orientation of a subject during medical imaging described in the above-mentioned embodiments of the present application based on the image captured by the camera 850.

It should be noted that the medical imaging device 800 does not necessarily include all of the components shown in FIG. 8. In addition, the medical imaging device 800 may further include components not shown in FIG. 8, for which reference may be made to the related art.

Further provided in the embodiments of the present application is a computer-readable program which, when executed in a medical imaging system, causes a computer to execute, in the medical imaging system, the method for determining the orientation of a subject during medical imaging described in the foregoing embodiments.

Further provided in the embodiments of the present application is a storage medium having a computer-readable program stored therein, where the computer-readable program causes a computer to perform, in a medical imaging system, the method for determining the orientation of a subject during medical imaging described in the foregoing embodiments.

The above apparatus and method of the present application can be implemented by hardware, or can be implemented by hardware in combination with software. The present application relates to the foregoing type of computer-readable program. When executed by a logic component, the program causes the logic component to implement the foregoing apparatus or a constituent component, or causes the logic component to implement various methods or steps as described above. The present application further relates to a storage medium for storing the above program, such as a hard disk, a disk, an optical disk, a DVD, a flash memory, etc.

The method/apparatus described in view of the embodiments of the present application may be directly embodied as hardware, a software module executed by a processor, or a combination of the two. For example, one or more of the functional block diagrams and/or one or more combinations of the functional block diagrams shown in the drawings may correspond to either respective software modules or respective hardware modules of a computer program flow. The foregoing software modules may respectively correspond to the steps shown in the figures. The foregoing hardware modules can be implemented, for example, by firming the software modules using a field-programmable gate array (FPGA).

The software modules may be located in a RAM, a flash memory, a ROM, an EPROM, an EEPROM, a register, a hard disk, a portable storage disk, a CD-ROM, or any other form of storage medium known in the art. The storage medium may be coupled to a processor, so that the processor can read information from the storage medium and can write information into the storage medium. Alternatively, the storage medium may be a constituent component of the processor. The processor and the storage medium may be located in an ASIC. The software module may be stored in a memory of a mobile terminal, and may also be stored in a memory card that can be inserted into a mobile terminal. For example, if a device (such as a mobile terminal) uses a large-capacity MEGA-SIM card or a large-capacity flash memory device, the software modules can be stored in the MEGA-SIM card or the large-capacity flash memory apparatus.

One or more of the functional blocks and/or one or more combinations of the functional blocks shown in the accompanying drawings may be implemented as a general-purpose processor, a digital signal processor (DSP), an application-specific integrated circuit (ASIC), a field-programmable gate array (FPGA) or other programmable logic devices, discrete gate or transistor logic devices, a discrete hardware assembly, or any appropriate combination thereof for implementing the functions described in the present application. The one or more functional blocks and/or the one or more combinations of the functional blocks shown in the accompanying drawings may also be implemented as a combination of computing devices, such as a combination of a DSP and a microprocessor, multiple microprocessors, one or more microprocessors in communication combination with a DSP, or any other such configuration.

The present application is described above with reference to specific embodiments. However, it should be clear to those skilled in the art that the foregoing description is merely illustrative and is not intended to limit the scope of protection of the present application. Various variations and modifications may be made by those skilled in the art according to the principle of the present application, and said variations and modifications also fall within the scope of the present application.

1. A method for determining the orientation of a subject during medical imaging, the method being characterized by comprising:
acquiring an image sequence of a subject captured by a camera, the image sequence comprising a plurality of images based on a time sequence;
determining, based on the image sequence, whether the state of the subject is abnormal; and
in response to a determination result that the state of the subject is abnormal, determining the orientation of the subject by using a first orientation or a second orientation,
wherein the first orientation is the orientation of the subject determined based on an image before a period when the state of the subject is determined to be abnormal, and the second orientation is the orientation of the subject determined based on an image after the period when the state of the subject is determined to be abnormal.

2. The method according to claim 1, wherein
determining whether the state of the subject is abnormal comprises:
determining at least one of whether the subject is blocked and whether the subject is moving.

3. The method according to claim 2, wherein
in response to a determination result that the subject is blocked, determining the orientation of the subject by using the first orientation.

4. The method according to claim 3, wherein
determining the orientation of the subject by using the first orientation comprises:
taking, as the orientation of the subject, the first orientation corresponding to a first predetermined image before the state of the subject is determined to be blocked; or
performing processing on the first orientations respectively corresponding to at least two images before the state of the subject is determined to be blocked, and obtaining the orientation of the subject.

5. The method according to claim 4, wherein the processing comprises:
performing processing of weighted summation or averaging on the first orientations respectively corresponding to the at least two images.

6. The method according to claim 4, wherein the processing comprises:
processing of selecting the first orientation the number of occurrences of which is the greatest or selecting the first orientation the number of occurrences of which exceeds a threshold.

7. The method according to claim 2, wherein
in response to a determination result that the subject is moving, the orientation of the subject is determined by using the second orientation.

8. The method according to claim 7, wherein
determining the orientation of the subject by using the second orientation comprises:
taking, as the orientation of the subject, the second orientation corresponding to a second predetermined image after the period when the state of the subject is determined to be moving; or
performing processing on the second orientations respectively corresponding to at least two images after the period when the state of the subject is determined to be moving, and obtaining the orientation of the subject.

9. The method according to claim 8, wherein the processing comprises:
performing processing of weighted summation or averaging on the second orientations respectively corresponding to the at least two images.

10. The method according to claim 8, wherein the processing comprises:
processing of selecting the second orientation the number of occurrences of which is the greatest or selecting the second orientation the number of occurrences of which exceeds a threshold.

11. A medical imaging system, comprising a memory and a processor, the memory storing a computer program, and the processor being configured to execute the computer program so as to implement the method for determining the orientation of a subject during medical imaging according to claim 1.

12. An apparatus for determining the orientation of a subject during medical imaging, the apparatus comprising:
an acquisition unit, which acquires an image sequence of a subject captured by a camera, the image sequence comprising a plurality of images based on a time sequence;
a determination unit, which determines, based on the image sequence, whether the state of the subject is abnormal; and
an orientation determination unit, which in response to a determination result that the state of the subject is abnormal, determines the orientation of the subject by using a first orientation or a second orientation,
wherein the first orientation is the orientation of the subject determined based on an image before a period when the state of the subject is determined to be abnormal, and the second orientation is the orientation of the subject determined based on an image after the period when the state of the subject is determined to be abnormal.
